(19) United States
(12) Patent Application Publication
(10) Pub. No.: US 2026/0357278 A1
(43) Pub. Date: Nov. 12, 2026

(54) POLYKETIDE SYNTHASE AND A TRANSGENIC CELL, TISSUE, AND ORGANISM COMPRISING SAME

(71) Applicant: YEDA RESEARCH AND DEVELOPMENT CO. LTD., Rehovot (IL)

(72) Inventors: Asaph AHARONI, Tel Aviv (IL); Prashant SONAWANE, Rehovot (IL); Adam JOZWIAK, Rehovot (IL); Paula BERMAN, Petach Tikva (IL); Luis DE-HARO, Rehovot (IL)

(21) Appl. No.: 19/109,194

(22) PCT Filed: Sep. 7, 2023
(86) PCT No.: PCT/IL2023/050969

§ 371 Date: Mar. 6, 2025

Related U.S. Application Data
(60) Provisional application No. 63/404,604, filed on Sep. 8, 2022.

(51) Int. Cl. C12N 15/82; C12N 1/20; C12N 9/10; C12P 17/06; C12R 1/01

(52) U.S. Cl. CPC C12N 15/8243; C12N 1/20; C12N 9/1029; C12N 15/8205; C12P 17/06; C12N 2800/10; C12R 2001/01

(57) ABSTRACT

The present invention provides polynucleotide sequences derived from Helichrysum umbraculigerum and encoding a protein or a plurality thereof belonging to the polyketide synthase (PKS) family. Further provided are an artificial nucleic acid molecule including the polynucleotide, a transgenic cell, tissue, or plant including same. Further provided are method for synthesizing a polyketide.

### CROSS-REFERENCE TO RELATED APPLICATIONS

This application claims the benefit of priority of U.S. Provisional Patent Application No. 63/404,604, titled “POLYKETIDE SYNTHASE AND A TRANSGENIC CELL, TISSUE, AND ORGANISM COMPRISING SAME”, filed 8 Sep. 2022, the contents of which are incorporated herein by reference in their entirety.

### REFERENCE TO AN ELECTRONIC SEQUENCE LISTING

The contents of the electronic sequence listing (YEDA-P-005-PCT ST26.xml; size: 14,356 bytes; and date of creation: Aug. 31, 2023) is herein incorporated by reference in its entirety.

### FIELD OF INVENTION

The present invention relates to polyketide synthesizing enzymes (PKS) including polynucleotides encoding same, and methods of using same, such as for producing a polyketide.

### BACKGROUND

Polyketides are a large, structurally diverse family of natural products. Polyketides possess a broad range of biological activities including antibiotic and pharmacological properties.

For example, polyketides are represented by such antibiotics as tetracyclines and erythromycin, anticancer agents including daunomycin, immunosuppressants, for example FK506 and rapamycin, and veterinary products such as monensin and avermectin.

Polyketides occur in most groups of organisms and are especially abundant in a class of mycelial bacteria, the actinomycetes, which produce various polyketides. Polyketide synthases (PKSs) are multifunctional enzymes related to fatty acid-1-synthases (FASs). PKSs catalyze the biosynthesis of polyketides through repeated (decarboxylative) Claisen condensations between acylthioesters, usually acetyl, propionyl, malonyl or methylmalonyl. Following each condensation, they introduce structural variability into the product by catalyzing all, part, or none of a reductive cycle comprising a ketoreduction, dehydration, and enoylreduction on the β-keto group of the growing polyketide chain. PKSs incorporate enormous structural diversity into their products, in addition to varying the condensation cycle, by controlling the overall chain length, choice of primer and extender units and, particularly in the case of aromatic polyketides, regiospecific cyclizations of the nascent polyketide chain. After the carbon chain has grown to a length characteristic of each specific product, it is released from the synthase by thiolysis or acyltransfer. Thus, PKSs consist of families of enzymes which work together to produce a given polyketide. It is the controlled variation in chain length, choice of chain-building units, and the reductive cycle, genetically programmed into each PKS, that contributes to the variation seen among naturally occurring polyketides. Two general classes of PKSs exist. These classifications are well known.

Polyketide synthases are known to be involved in cannabinoid biosynthesis.

Genes encoding enzymes of cannabinoid biosynthesis can also be useful in synthesis of cannabinoid analogs and synthesis of analogs of cannabinoid precursors. Cannabinoid analogs have been previously synthesized and may be useful as pharmaceutical products. There remains a need in the art to identify enzymes, and nucleotide sequences encoding such enzymes, that are involved in the synthesis of aromatic polyketides.

### SUMMARY

According to a first aspect there is provided an isolated DNA molecule comprising a nucleic acid sequence having at least 83% homology to any one of SEQ ID Nos: 1-4, or any combination thereof.

According to another aspect, there is provided an artificial nucleic acid molecule comprising a nucleic acid sequence having at least 83% homology to any one of SEQ ID Nos: 1-4, or any combination thereof.

According to another aspect, there is provided a plasmid or an agrobacterium comprising a nucleic acid sequence having at least 83% homology to any one of SEQ ID Nos: 1-4, or any combination thereof.

According to another aspect, there is provided an isolated protein encoded by any one of: (a) the isolated DNA molecule of the invention; (b) the artificial vector disclosed herein; and the plasmid or agrobacterium disclosed herein.

According to another aspect, there is provided a transgenic cell comprising: (a) a nucleic acid sequence having at least 83% homology to any one of SEQ ID Nos: 1-4, or any combination thereof; (b) the artificial nucleic acid molecule disclosed herein; (b) the plasmid or agrobacterium disclosed herein; (c) the isolated protein disclosed herein; or (d) any combination of (a) to (d).

According to another aspect, there is provided an extract derived from the transgenic cell of disclosed herein, or any fraction thereof.

According to another aspect, there is provided a transgenic plant, a transgenic plant tissue or a plant part, comprising: (a) a nucleic acid sequence having at least 83% homology to any one of SEQ ID Nos: 1-4, or any combination thereof; (b) the artificial vector disclosed herein; (b) the plasmid or agrobacterium disclosed herein; (c) the isolated protein disclosed herein; (d) the transgenic cell disclosed; or (e) any combination of (a) to (e).

According to another aspect, there is provided a composition comprising: (a) the isolated DNA molecule of the invention; (b) the artificial vector disclosed herein; (c) the plasmid or agrobacterium disclosed herein; (d) the isolated protein disclosed herein; (e) the transgenic cell disclosed herein; (f) the extract disclosed herein; (g) the transgenic plant tissue or plant part disclosed herein; or (h) any combination of (a) to (g), and an acceptable carrier.

According to another aspect, there is provided a method for synthesizing a polyketide comprising the steps: (a) providing a cell comprising an artificial vector comprising a nucleic acid sequence having at least 83% homology to any one of: SEQ ID Nos: 1-4; and (b) culturing the cell from step (a) such that a protein encoded by the artificial vector is expressed, thereby synthesizing a polyketide.

According to another aspect, there is provided a method for synthesizing a polyketide comprising contacting a diketide substrate with an effective amount of protein comprising an amino acid sequence with at least 93% homology to any one of SEQ ID Nos: 5-8, thereby synthesizing a polyketide.

According to another aspect, there is provided a method for obtaining an extract from a transgenic cell or a transfected cell comprising the steps: (a) culturing a transgenic cell or a transfected cell in a medium, wherein the transgenic cell or the transfected cell comprises a nucleic acid sequence having at least 83% homology to any one of SEQ ID Nos: 1-4; and (b) extracting the transgenic cell or the transfected cell, thereby obtaining an extract from the transgenic cell or the transfected cell.

According to another aspect, there is provided an extract of a transgenic cell or a transfected cell obtained according to the herein disclosed method.

According to another aspect, there is provided a medium or a portion thereof separated from a cultured transgenic cell or a cultured transfected cell, obtained according to the herein disclosed method.

According to another aspect, there is provided a composition comprising: (a) the extract disclosed herein; (b) the medium disclosed herein or a portion thereof; or (c) a combination of (a) and (b), and an acceptable carrier.

In some embodiments, the nucleic acid sequence has at least 83% homology to any one of SEQ ID Nos: 1-4 is 1,000 to 1,400 nucleotides long.

In some embodiments, the nucleic acid sequence encodes a protein being a polyketide synthase.

In some embodiments, the isolated protein comprises an amino acid sequence with at least 93% homology to any one of SEQ ID Nos: 5-8.

In some embodiments, the isolated protein consists of an amino acid sequence of any one of SEQ ID Nos: 5-8.

In some embodiments, the isolated protein is characterized by having an activity of polymerizing a diketide substrate into a polyketide.

In some embodiments, the diketide substrate is obtained by coupling of an acyl CoA starting unit.

In some embodiments, the acyl CoA starting unit is selected form the group consisting of: acetyl COA, butyryl CoA, hexanoyl CoA, octanoyl CoA, cinnamoyl CoA, coumaroyl CoA, and any combination thereof.

In some embodiments, the acyl CoA is hexanoyl CoA, cinnamoyl CoA, or both.

In some embodiments, the polyketide comprises a tetraketide.

In some embodiments, the transgenic cell is any one of: a unicellular organism, a cell of a multicellular organism, and a cell in a culture.

In some embodiments, the unicellular organism comprises a fungus or a bacterium.

In some embodiments, the fungus is a yeast cell.

In some embodiments, the extract comprises the isolated DNA molecule, the isolated protein, or both.

In some embodiments, the transgenic plant is a Cannabis sativa plant.

In some embodiments, the protein is characterized by having an activity of polymerizing a diketide substrate into a polyketide.

In some embodiments, the culturing comprises supplementing the cell with an effective amount of a diketide substrate, an acyl CoA starting unit, or both.

In some embodiments, the artificial vector is an expression vector.

In some embodiments, the cell is a prokaryote cell or a eukaryote cell.

In some embodiments, the cell is a transgenic cell, or a cell transfected with the isolated DNA molecule of the invention or the artificial vector disclosed herein.

In some embodiments, the method further comprises a step preceding step (a), comprising introducing or transfecting the cell with the artificial vector.

In some embodiments, contacting is in a cell-free system.

In some embodiments, the method further comprises a step preceding step (b), comprising separating the cultured transgenic cell or the cultured transfected cell from the medium.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-1B include a scheme and graphs of ion abundances. (1A) A scheme presenting the steps and types of by-products produced in in-vitro recombinant enzymatic assays with Helichrysum umbraculigerum polyketide synthase (HuPKS) with or without Cannabis sativa olivetolic acid cyclase (CsOAC). (1B) Peak areas of ion abundances of products following in-vitro recombinant enzymatic assays with or without CsOAC. PKS, polyketide synthase; PDAL, pentyl diacetic acid lactone; HTAL, triacetic acid lactone; OA, olivetolic acid; THPH, hexanoylphloroglucinol; EV, crude proteins obtained from E. coli cells transformed with empty vector.

FIGS. 2A-2B include extracted ion chromatograms and tandem mass spectrometry (MS/MS) spectra showing the products of recombinant enzyme assays of HuPKS4 with either an empty vector (EV, crude proteins obtained from E. coli cells transformed with empty vector) or CsOAC, in the presence of hexanoyl-CoA and malonyl-CoA. (2A) Extracted ion chromatograms show the formation of olivetol ([M+H]+=181.123 Da), pentyl diacetic acid lactone (PDAL; [M−H]−=181.087 Da), hexanoyl triacetic acid lactone (HTAL), olivetolic acid (OA) and hexanoylphloroglucinol (THPH) ([M−H]−=223.097 Da). Olivetol, OA and THPH standards are shown for reference. Magnification of the extracted ion chromatograms normalized to the maximum value in each plot is shown for improved interpretation. (2B) MS/MS spectra of the produced OA compared to the standard (Std).

### DETAILED DESCRIPTION

The present invention, in some embodiments, is directed to polynucleotide sequences derived from Helichrysum umbraculigerum and encoding a protein or a plurality thereof belonging to the polyketide synthase (PKS) family.

According to some embodiments, there is provided a polynucleotide comprising a nucleic acid sequence comprising any one of SEQ ID Nos: 1-4, or any combination thereof (“polynucleotide of the invention”).

In some embodiments, the polynucleotide is an isolated polynucleotide. In some embodiments, the polynucleotide is a DNA molecule. In some embodiments, the polynucleotide is an isolated DNA molecule. In some embodiments, the DNA molecule is an isolated DNA molecule. In some embodiments, the DNA molecule is a complementary DNA (cDNA) molecule.

As used herein, the terms “isolated polynucleotide” and “isolated DNA molecule” refers to a nucleic acid molecule that is essentially free from contaminating cellular components, such as carbohydrate, lipid, or other proteinaceous impurities associated with the nucleic acid in nature. Typically, a preparation of isolated DNA or RNA contains the nucleic acid in a highly purified form, e.g., at least about 80% pure, at least about 90% pure, at least about 95% pure, greater than 95% pure, or greater than 99% pure. In some embodiments, the isolated polynucleotide is any one of DNA, RNA, and cDNA. In some embodiments, the isolated polynucleotide is a synthesized polynucleotide. Synthesis of polynucleotides is well known in the art and may be performed, for example, by ligating or covalently linking by primer linkers multiple nucleic acid molecules together.

The term “nucleic acid” is well known in the art. A “nucleic acid” as used herein will generally refer to any molecule (e.g., a strand) of DNA, RNA or a derivative or analog thereof, comprising nucleotides. Nucleotides are comprised of nucleosides and phosphate groups. The nitrogenous bases of nucleosides include, for example, naturally occurring purine or pyrimidine nucleosides as found in DNA (e.g., an adenine “A,” a guanine “G,” a thymine “T” or a cytosine “C”) or RNA (e.g., an A, a G, an uracil “U” or a C).

The term “nucleic acid molecule” includes but is not limited to single-stranded RNA (ssRNA), double-stranded RNA (dsRNA), single-stranded DNA (ssDNA), double-stranded DNA (dsDNA), small RNAs, circular nucleic acids, fragments of genomic DNA or RNA, degraded nucleic acids, amplification products, modified nucleic acids, plasmid or organellar nucleic acids, and artificial nucleic acids such as oligonucleotides.

In some embodiments, the polynucleotide comprises or consists of the nucleic acid sequence:

(SEQ ID NO: 1)
ATGGCATCCTCAATTAATATCTCCAAGATCAGAGAGGCTCAACGAGCACA
AGGTCCAGCCTCTATTCTTGCTGTCGGTACCGCGAATCCGTCTAATTGCG
TGTATCAAGCTGATTATCCTGATTACTACTTTCGAATCACTAAAAGTGAA
CACATGGTTGATCTCAAACGGAAATTCAAGCGCATGTGTGACCAATCTAT
GATAAGAAAGCGGTACATGCAAATTACGGAGGAGTATCTGAAAGAAAACC
CCAACATTTGTGAATACATGGCTCCATCACTTGACGCCCGTCAAGACGTT
GTAGTCGTCGAAGTCCCAAAACTCGGTAAAGAAGCCGCAACAAAAGCCAT
CAAAGAATGGGGCCAACCAAAATCCAAAATTACCCATCTCATCTTTTGTA
CCACGTCCGGTGTCGACATGCCCGGAGCAGATTACCAGCTCACCAAACTC
CTCGGTCTTTGTCCTTCAGTCAAACGCTTTATGATGTACCAACAAGGTTG
TTTTGCTGGTGGCACGGTTCTTCGTCTAGCTAAGGACATCGCTGAGAACA
ATAAAGGTGCTCGTGTACTTGTCGTTTGTTCCGAGATTACAGCTGTCATT
TTTCGTGGACCCAACGACACTCACCTTGATTCACTTATCGGTCAAGCGTT
ATTTGGGGATGGGGCATCTTCGGTTATCGTGGGGTCTGACCCAGACTTGA
CAACCGAGCGGCCATTGTTTGAAATCATATCGGCTGCACAAACGATTTTA
CCGGACTCTGAAGGTGCGATAGATGGACACTTGAGGGAAGCTGGGTTAAC
TTTTCATCTACTTAAAGACGTACCGAGGTTGATTTCGAAGAATATAGAGA
AAGCTTTAACACAAGCATTTTCTCCCCTGGGAATTAGTGACTGGAACTCT
ATCTTTTGGGTCACGCACCCTGGTGGTCCAGCTATACTGGACCAAGTGGA
ACTCAAACTTGGACTCAAAGAGGAGAAGATGAGAACCACTAGACATGTTC
TCAGTGAATATGGGAACATGTCTAGTGCATGTGTTTTTTTTGTACTTGAT
GAAATGAGAAAGAGATCGGCTAAAGGCGGTGCGAGGACCACCGGAGAAGG
GTTAGATTGGGGTGTTCTGTTTGGGTTTGGTCCGGGTTTAACGGTTGAGA
CTGTGGTCCTTCATAGTCTCCCAACTACTATGTCGATTGCGACTTAA

In some embodiments, the polynucleotide comprises a nucleic acid sequence with at least 83%, at least 85%, at least 87%, at least 95%, or at least 99% homology or identity to SEQ ID NO: 1, or any value and range therebetween. Each possibility represents a separate embodiment of the invention. In some embodiments, the polynucleotide comprises a nucleic acid sequence with 83% to 100%, 88% to 100%, 90% to 100%, or 95% to 100% homology or identity to SEQ ID NO: 1. Each possibility represents a separate embodiment of the invention.

In some embodiments, the polynucleotide comprises or consists of the nucleic acid sequence:

(SEQ ID NO: 2)
ATGGCATCCTCAATTAATATCTCCAAGATCAGAGAGGCTCAACGAGCACA
AGGTCCAGCCTCTATTCTTGCTGTCGGTACTGCGAATCCGTCTAATTGTG
TGTATCAAGCTGATTATCCTGATTACTACTTTCGAATCACTAAAAGTGAA
CACATGGTTGATTTGAAAGAGAAATTCCAGCGCATGTGTGACAAATCTAT
GATAAGAAAGCGGCACATTCACATTACGGAGGAGTTTTTGAAAGAAAACC
CAAACCTTTGTGAATACATGGCTCCATCACTTGACACCCGTCAAGACGTT
GTAGTCGTCGAAGTCCCAAAACTCGGTAAAGAAGCCGCAACAAAAGCCAT
CAAAGAATGGGGCCAACCAAAATCCAAAATTACCCATCTCATCTTTTGTA
CCACGTCCGGTGTCGACATGCCCGGAGCAGATTACCAGCTCACCAAACTC
CTCGGTCTCCATCCTTCAGTCAAACGCTTTATGATGTACCAACAAGGTTG
TTTTGCTGGTGGCACGGTTCTTCGTCTAGCTAAGGACCTCGCTGAGAACA
ATAAAGGTGCTCGTGTACTTGCCGTTTGTTCCGAGATTACAGCTGTCACG
TTTCGTGGACCCAACGACACTCACATTGATTCACTTGTCGGTCAAGCATT
ATTTGGGGACGGGGCAGCTGCGGTTATCGTGGGGTCTGATCCTGACTTGA
CAACTGAGCGGCCGTTGTTTGAAATCATATCGGCTGCACAAACGATTTTA
CCGAACTCTGAAGGTGCGATAGATGGACATGTGAGGGAAGTTGGGGTAAC
TATTCATATACTTAAAGACGTCCCGGTGTTGATTTCGAAGAATATAGAGA
AAGCTTTAACACAAGCATTTTCTCCCTTAGGAATTAGTGACTGGAACTCG
ATCTTTTGGGTCGTACACCCTGGTGGTCCAGCTATACTGGACCAAGTGGA
ACTCAAACTTGGACTCAAAGAGGAGAAAATGAGAACCACTAGACATGTTC
TCAGTGAATATGGGAACATGTCTAGTGCATGTGTTTTTTTTGTACTTGAT
GAAATGAGAAAGAGATCGGCTAAAGGCGGTGCGAGGACCACCGGAGAAGG
GTTAGATTGGGGTGTTCTGTTTGGGTTTGGTCCAGGTTTAACGGTTGAGA
CGGTGGTCCTTCATAGTCTCCCAACTACTATGTCGATTGCAACTTAA.

In some embodiments, the polynucleotide comprises a nucleic acid sequence with at least 83%, at least 85%, at least 87%, at least 89%, at least 92%, at least 95%, at least 97%, or at least 99% homology or identity to SEQ ID NO: 2, or any value and range therebetween. Each possibility represents a separate embodiment of the invention. In some embodiments, the polynucleotide comprises a nucleic acid sequence with 83% to 100%, 87% to 100%, 90% to 100%, or 93% to 100% homology or identity to SEQ ID NO: 2. Each possibility represents a separate embodiment of the invention.

In some embodiments, the polynucleotide comprises or consists of the nucleic acid sequence:

(SEQ ID NO: 3)
ATGGCATCCTCAATTAATATCTCCAAGATCAGAGAGGCTCAACGAGCACA
AGGTCCAGCCTCTATTCTTGCTGTCGGTACCGCGAATCCGTCTAATTGCG
TGTATCAAGCTGATTATCCTAATTACTACTTTCGAATCACTAAAAGTGAA
CACATGGTTGATCTCAAACGGAAATTCAAGCGCATGTGTGACCAATCTAT
GATAAGAAAGCGGTACATGCAAATTACGGAGGAGTATCTGAAAGAAAACC
CCAACATTTGTGAATACATGGCTCCATCACTTGACGCCCGTCAAGACGTT
GTAGTCGTCGAAGTCCCAAAACTCGGTAAAGAAGCCGCAACAAAAGCCAT
CAAAGAATGGGGCCAACCAAAATCCAAAATTACCCATCTCATCTTTTGTA
CCACGTCCGGTGTCGACATGCCCGGAGCAGATTACCAGCTCACCAAACTC
CTCGGTCTCTGTCCTTCAGTCAAACGCTTTATGATGTACCAACAAGGTTG
TTTTGCTGGTGGCACGGTTCTTCGTCTAGCTAAGGACATCGCTGAGAACA
ATAAAGGTGCTCGTGTACTTGTCGTTTGTTCCGAGATTACAGCTGTCATT
TTTCGTGGACCCAACGACACTCACCTTGATTCACTTATCGGTCAAGCGTT
ATTTGGGGATGGGGCATCTTCGGTTATCGTGGGGTCTGACCCAGACTTGA
CAACCGAGCGGCCATTGTTTGAAATCATATCGGCTGCACAAACGATTTTA
CCGGACTCTGAAGGTGCGATAGATGGACACTTGAGGGAAGCTGGGTTAAC
TTTTCATCTACTTAAAGACGTACCGGGGTTGATTTCGAAGAATATAGAGA
AAGCTTTAACACAAGCATTTTCTCCCTTGGGAATTAGTGACTGGAACTCT
ATCTTTTGGGTCACGCACCCTGGTGGTCCAGCTATACTGGACCAAGTGGA
ACTCAAACTTGGACTCAAAGAGGAGAAGATGAGAGCCTCTAGACATGTTC
TCAGTGAATACGGGAACATGTCTAGTGCATGTGTTTTTTTTATACTTGAT
GAAATGAGAAAGAAATCGGATGAAGATGGTGCGCCGACCACTGGAGAAGG
GTTAGATTGGGGTGTTCTGTTTGGGTTTGGTCCGGGTTTAACGGTTGAGA
CGGTGGTCCTTCATAGTCTCCCAACTACTATGTCGATTGCGACTTAA.

In some embodiments, the polynucleotide comprises a nucleic acid sequence with at least 83%, at least 87%, at least 89%, at least 90%, at least 92%, at least 95%, at least 97%, or at least 99% homology or identity to SEQ ID NO: 3, or any value and range therebetween. Each possibility represents a separate embodiment of the invention. In some embodiments, the polynucleotide comprises a nucleic acid sequence with 83% to 100%, 88% to 100%, 93% to 100%, or 95% to 100% homology or identity to SEQ ID NO: 3. Each possibility represents a separate embodiment of the invention.

In some embodiments, the polynucleotide comprises or consists of the nucleic acid sequence:

(SEQ ID NO: 4)
ATGGCATCCTCAATTAATATCTCTAAGATCAGAGAGGCTCAACGAGCACA
AGGTCCAGCCTCTATTCTTGCTGTCGGTACTGCGAATCCATCTAATTATG
AGATTCAAGCTGATTTTCCTGATTACTACTTTCGAGTCACTAAAAGTGAA
CACATGGCTGATATGAAAGGGACATTCCAGCGCATGTGTGACAAATCTAT
GATAAGAAAGCGGCACATGCTCATTACGGAGGAGTTTTTGAAAGAAAACC
CAAACCTTTGTGAATACATGGCTCCATCACTTGACACCCGTCAAGACGTT
GTAGTCGTCGAAGTCCCAAAACTCGGTAAAGAAGCCGCAACAAAAGCCAT
CAAAGAATGGGGCCAACCAAAATCCAAAATTACCCATCTCATCTTTTGTA
CTACAACTGGTGTCGACATGCCTGGAGCCGATTACCAGCTCACCAAGCTC
CTCGGCCTCGCTCCTTCAGTCAAACGCTTTATGATATACCAACAAGGTTG
TTTTGCTGGTGGCACGGTTCTTCGTCTTGCTAAAGACATAGCTGAGAACA
ATAAAGGTGCTCGTGTACTTGCCGTATGTTCAGAGATTACAGCTATGTCG
TTTCGTGGGCCCAATGACACTCACGTTGATTCACTTGTCGGTCAAGCATT
ATTTGGGGACGGGGCAGCTGCAGTTATCGTGGGGTCTGATCCTGACTTGA
CAACCGAGCGGCCGTTGTTTGAAATCATATCGGCTGCACAAACGATTTTA
CCAAACTCTGAAGGTGCGATAGATGGACATGTGAGGGAAGTTGGTTTAAC
TATTCATATACTTAAAGACGTCCCGGTGTTGATATCGAAGAATATAGAGA
AAGCTTTGACACAAGCATTTTCTCCCTTAGGAATTAGTGACTGGAACTCG
ATCTTTTGGATCGTACACCCTGGTGGTCCAGCTATACTGGACCAAGTGGA
ACTCAAAGTTGGACTCAAAAAGGAGAAAATGGCAACCAGTAGACATGTTC
TAAGTGAATACGGGAACATGTCTAGTGCATGTGTTTTTTTTATAATGGAT
GAAATGAGAAAGAGATCGGCTAAAGGCGGTGCGAGGACCACCGGAGAAGG
GTTAGATTGGGGTGTTTTGTTTGGGTTTGGTCCAGGTTTAACGGTTGAGA
CGGTGGTCCTTCATAGTCTCCCAACTACAATGTAG.

In some embodiments, the polynucleotide comprises a nucleic acid sequence with at least 82%, at least 85%, at least 89%, at least 92%, at least 95%, at least 97%, or at least 99% homology or identity to SEQ ID NO: 4, or any value and range therebetween. Each possibility represents a separate embodiment of the invention. In some embodiments, the polynucleotide comprises a nucleic acid sequence with 82% to 100%, 86% to 100%, 90% to 100%, or 95% to 100% homology or identity to SEQ ID NO: 4. Each possibility represents a separate embodiment of the invention.

In some embodiments, the polynucleotide of the invention comprises 1,000 to 1,400 nucleotides. In some embodiments, the polynucleotide of the invention is 1,000 to 1,400 nucleotides long.

In some embodiments, 1,000 to 1,400 nucleotides comprises: at least 1,000 nucleotides, at least 1,200 nucleotides, at least 1,250 nucleotides, at least 1,300 nucleotides, at least 1,350 nucleotides, at least 1,370 nucleotides, or at least 1,390 nucleotides, or any value and range therebetween. Each possibility represents a separate embodiment of the invention. In some embodiments, 1,000 to 1,400 nucleotides comprises: 1,000 to 1,050 nucleotides, 1,000 to 1,175 nucleotides, 1,000 to 1,250 nucleotides, 1,000 to 1,300 nucleotides, 1,000 to 1,350 nucleotides, 1,000 to 1,370 nucleotides, or 1,000 to 1,390 nucleotides. Each possibility represents a separate embodiment of the invention.

In some embodiments, the polynucleotide comprises a plurality of polynucleotides. In some embodiments, the polynucleotide comprises a plurality of types of polynucleotides. As used herein, the term “plurality” comprises any integer equal to or greater than 2. In some embodiments, the polynucleotide comprises at least 2, or at least 3 different nucleic acid sequences, or any value and range therebetween, wherein each of the different nucleic acid sequences is selected from SEQ ID Nos: 1-4. Each possibility represents a separate embodiment of the invention. In some embodiments, the polynucleotide comprises 2-3, 2-4, or 3-4 different nucleic acid sequences, wherein each of the different nucleic acid sequences is selected from SEQ ID Nos: 1-4.

In some embodiments, the polynucleotide comprises a plurality of polynucleotide molecules, wherein each of the plurality of the polynucleotide molecules comprises a different nucleic acid sequence, and wherein the different nucleic acid sequences are selected from SEQ ID Nos.: 1-4.

In some embodiments, the polynucleotide encodes a protein characterized by polyketide synthesizing activity. In some embodiments, the polynucleotide encodes a protein being a polyketide synthase (PKS). In some embodiments, the PKS is a PKS derived from Helichrysum umbraculigerum. As used herein, the terms “polyketide synthase” and “PKS” encompasses any enzyme derived from H. umbraculigerum and having or characterized by being functional analog of the “olivetol synthase” or “OLS” of Cannabis sativa.

As used herein, the terms “polyketide synthase” and “PKS” are interchangeable, and refer to any peptide, polypeptide, or a protein, capable of catalyzing the elongation of a ketide or a polyketide chain. In some embodiments, PKS activity transacylation. In some embodiments, PKS activity comprises Claisen condensation. In some embodiments, PKS activity comprises reduction of β-keto group to a β-hydroxy group. In some embodiments, PKS activity comprises H₂O splitting, thereby obtaining, providing, or resulting in a α-β-unsaturated alkene. In some embodiments, PKS activity comprises reducing a α-β-double-bond to a single-bond. In some embodiments, PKS activity comprises hydrolyzing a polyketide chain or a completed polyketide chain from an acyl carrier protein domain of the PKS. In some embodiments, PKS activity comprises polymerizing and/or ligating a diketide substrate into a polyketide chain. In some embodiments, PKS activity comprises elongating a diketide to a polyketide chain. In some embodiments, PKS activity comprises elongating a polyketide chain.

According to some embodiments, there is provided an artificial nucleic acid molecule comprising the polynucleotide disclosed herein.

In some embodiments, the artificial vector comprises a plasmid. In some embodiments, the artificial vector comprises or is an agrobacterium comprising the artificial nucleic acid molecule. In some embodiments, the artificial vector is an expression vector. In some embodiments, the artificial vector is a plant expression vector. In some embodiments, the artificial vector is for use in expressing a PKS encoding nucleic acid sequence as disclosed herein. In some embodiments, the artificial vector is for use in heterologous expression of a PKS encoding nucleic acid sequence as disclosed herein in a cell, a tissue, or an organism. In some embodiments, the artificial vector is for use in producing or the production of a polyketide in a cell, a tissue, or an organism.

Expressing of a polynucleotide within a cell is well known to one skilled in the art. It can be carried out by, among many methods, transfection, viral infection, or direct alteration of the cell's genome. In some embodiments, the polynucleotide is in an expression vector such as plasmid or viral vector. A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

The vector may be a DNA plasmid delivered via non-viral methods or via viral methods. The viral vector may be a retroviral vector, a herpesviral vector, an adenoviral vector, an adeno-associated viral vector, a virgaviridae viral vector, or a poxviral vector. The barley stripe mosaic virus (BSMV), the tobacco rattle virus and the cabbage leaf curl geminivirus (CbLCV) may also be used. The promoters may be active in plant cells. The promoters may be a viral promoter.

In some embodiments, the polynucleotide as disclosed herein is operably linked to a promoter. The term “operably linked” is intended to mean that the nucleotide sequence of interest is linked to the regulatory element or elements in a manner that allows for expression of the nucleotide sequence (e.g., in an in vitro transcription/translation system or in a host cell when the vector is introduced into the host cell). In some embodiments, the promoter is operably linked to the polynucleotide of the invention. In some embodiments, the promoter is a heterologous promoter. In some embodiments, the promoter is the endogenous promoter.

In some embodiments, the vector is introduced into the cell by standard methods including electroporation (e.g., as described in From et al., Proc. Natl. Acad. Sci. USA 82, 5824 (1985)), heat shock, infection by viral vectors, high velocity ballistic penetration by small particles with the nucleic acid either within the matrix of small beads or particles, or on the surface (Klein et al., Nature 327. 70-73 (1987)), such as biolistic use of coated particles, and needle-like particles, Agrobacterium Ti plasmids and/or the like. The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins. The promoter may extend upstream or downstream of the transcriptional start site and may be any size ranging from a few base pairs to several kilo-bases.

In some embodiments, the polynucleotide is transcribed by RNA polymerase II (RNAP II and Pol II). RNAP II is an enzyme found in eukaryotic cells, known to catalyze the transcription of DNA to synthesize precursors of mRNA and most snRNA and microRNA.

In some embodiments, a plant expression vector is used. In one embodiment, the expression of a polypeptide coding sequence is driven by a number of promoters. In some embodiments, viral promoters such as the 35S RNA and 19S RNA promoters of CaMV [Brisson et al., Nature 310:511-514 (1984)], or the coat protein promoter to TMV [Takamatsu et al., EMBO J. 6:307-311 (1987)] are used. In another embodiment, plant promoters are used such as, for example, the small subunit of RUBISCO [Coruzzi et al., EMBO J. 3:1671-1680 (1984); and Brogli et al., Science 224:838-843 (1984)] or heat shock promoters, e.g., soybean hsp17.5-E or hsp17.3-B [Gurley et al., Mol. Cell. Biol. 6:559-565 (1986)]. In one embodiment, constructs are introduced into plant cells using Ti plasmid, Ri plasmid, plant viral vectors, direct DNA transformation, microinjection, electroporation and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-IMTHA, and vectors derived from Epstein Bar virus include pHEBO, and p205. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as systemic infection and targeting specificity, are used for in vivo expression. In one embodiment, systemic infection is inherent in the life cycle of, for example, the retrovirus and is the process by which a single infected cell produces many progeny virions that infect neighboring cells. In one embodiment, the result is that a large area becomes rapidly infected, most of which was not initially infected by the original viral particles. In one embodiment, viral vectors are produced that are unable to spread systemically. In one embodiment, this characteristic can be useful if the desired purpose is to introduce a specified gene into only a localized number of targeted cells.

In some embodiments, plant viral vectors are used. In some embodiments, a wild-type virus is used. In some embodiments, a deconstructed virus such as are known in the art is used. In some embodiments, Agrobacterium is used to introduce the vector of the invention into a virus.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation, agrobacterium Ti plasmids and infection with recombinant viral vectors. In addition, see U.S. Pat. Nos. 5,464,764 and 5,487,992 for positive-negative selection methods.

It will be appreciated that other than containing the necessary elements for the transcription and translation of the inserted coding sequence (encoding the polypeptide), the expression construct of the present invention can also include sequences engineered to optimize stability, production, purification, yield, or activity of the expressed polypeptide.

In some embodiments, the artificial vector comprises a polynucleotide encoding a protein comprising an amino acid sequence as described herein.

According to some embodiments, there is provided a protein encoded by: (a) the polynucleotide disclosed herein; (b) the artificial vector disclosed herein; or the plasmid or agrobacterium disclosed herein.

In some embodiments, the protein is encoded by a polynucleotide comprising or consisting of SEQ ID Nos: 1-4.

In some embodiments, the protein comprises an amino acid sequence with at least 88%, at least 90%, at least 93%, at least 95%, at least 97%, or at least homology or identity to any one of SEQ ID Nos: 5-8.

In some embodiments, the protein is an isolated protein.

As used herein, the terms “peptide”, “polypeptide” and “protein” are interchangeable and refer to a polymer of amino acid residues. In another embodiment, the terms “peptide”, “polypeptide” and “protein” as used herein encompass native peptides, peptidomimetics (typically including non-peptide bonds or other synthetic modifications) and the peptide analogues peptoids and semipeptoids or any combination thereof. In another embodiment, the peptides, polypeptides and proteins described have modifications rendering them more stable while in the organism or more capable of penetrating into cells. In one embodiment, the terms “peptide”, “polypeptide” and “protein” apply to naturally occurring amino acid polymers. In another embodiment, the terms “peptide”, “polypeptide” and “protein” apply to amino acid polymers in which one or more amino acid residue is an artificial chemical analogue of a corresponding naturally occurring amino acid.

As used herein, the terms “isolated protein” refers to a protein that is essentially free from contaminating cellular components, such as carbohydrate, lipid, or other proteinaceous impurities associated with the nucleic acid in nature. Typically, a preparation of an isolated protein contains the protein in a highly purified form, e.g., at least about 80% pure, at least about 90% pure, at least about 95% pure, greater than 95% pure, or greater than 99% pure. In some embodiments, the isolated protein is a synthesized protein. Synthesis of protein is well known in the art and may be performed, for example, by heterologous expression in a transformed cell, such as exemplified herein.

In some embodiments, the protein comprises or consists of the amino acid sequence:

(SEQ ID NO: 5)
MASSINISKIREAQRAQGPASILAVGTANPSNCVYQADYPDYYFRITKSE
HMVDLKRKFKRMCDQSMIRKRYMQITEEYLKENPNICEYMAPSLDARQDV
VVVEVPKLGKEAATKAIKEWGQPKSKITHLIFCTTSGVDMPGADYQLTKL
LGLCPSVKRFMMYQQGCFAGGTVLRLAKDIAENNKGARVLVVCSEITAVI
FRGPNDTHLDSLIGQALFGDGASSVIVGSDPDLTTERPLFEIISAAQTIL
PDSEGAIDGHLREAGLTFHLLKDVPRLISKNIEKALTQAFSPLGISDWNS
IFWVTHPGGPAILDQVELKLGLKEEKMRTTRHVLSEYGNMSSACVFFVLD
EMRKRSAKGGARTTGEGLDWGVLFGFGPGLTVETVVLHSLPTTMSIAT.

In some embodiments, the protein comprises an amino acid sequence with at least 92%, at least 96%, at least 98%, or at least 99% homology or identity to SEQ ID NO: 5, or any value and range therebetween. Each possibility represents a separate embodiment of the invention. In some embodiments, the protein comprises an amino acid sequence with 92% to 100%, 95% to 100%, 96% to 100%, or 98% to 100% homology or identity to SEQ ID NO: 5. Each possibility represents a separate embodiment of the invention.

In some embodiments, the protein comprises or consists of the amino acid sequence:

(SEQ ID NO: 6)
MASSINISKIREAQRAQGPASILAVGTANPSNCVYQADYPDYYFRITKSE
HMVDLKEKFQRMCDKSMIRKRHIHITEEFLKENPNLCEYMAPSLDTRQDV
VVVEVPKLGKEAATKAIKEWGQPKSKITHLIFCTTSGVDMPGADYQLTKL
LGLHPSVKRFMMYQQGCFAGGTVLRLAKDLAENNKGARVLAVCSEITAVT
FRGPNDTHIDSLVGQALFGDGAAAVIVGSDPDLTTERPLFEIISAAQTIL
PNSEGAIDGHVREVGVTIHILKDVPVLISKNIEKALTQAFSPLGISDWNS
IFWVVHPGGPAILDQVELKLGLKEEKMRTTRHVLSEYGNMSSACVFFVLD
EMRKRSAKGGARTTGEGLDWGVLFGFGPGLTVETVVLHSLPTTMSIAT.

In some embodiments, the protein comprises an amino acid sequence with at least 91%, at least 94%, at least 95%, or at least 97% homology or identity to SEQ ID NO: 6, or any value and range therebetween. Each possibility represents a separate embodiment of the invention. In some embodiments, the protein comprises an amino acid sequence with 91% to 100%, 94% to 100%, 97% to 100%, or 97% to 100% homology or identity to SEQ ID NO: 6. Each possibility represents a separate embodiment of the invention.

In some embodiments, the protein comprises or consists of the amino acid sequence:

(SEQ ID NO: 7)
MASSINISKIREAQRAQGPASILAVGTANPSNCVYQADYPNYYFRITKSE
HMVDLKRKFKRMCDQSMIRKRYMQITEEYLKENPNICEYMAPSLDARQDV
VVVEVPKLGKEAATKAIKEWGQPKSKITHLIFCTTSGVDMPGADYQLTKL
LGLCPSVKRFMMYQQGCFAGGTVLRLAKDIAENNKGARVLVVCSEITAVI
FRGPNDTHLDSLIGQALFGDGASSVIVGSDPDLTTERPLFEIISAAQTIL
PDSEGAIDGHLREAGLTFHLLKDVPGLISKNIEKALTQAFSPLGISDWNS
IFWVTHPGGPAILDQVELKLGLKEEKMRASRHVLSEYGNMSSACVFFILD
EMRKKSDEDGAPTTGEGLDWGVLFGFGPGLTVETVVLHSLPTTMSIAT.

In some embodiments, the protein comprises an amino acid sequence with at least 93%, at least 95%, or at least 97% homology or identity to SEQ ID NO: 7, or any value and range therebetween. Each possibility represents a separate embodiment of the invention. In some embodiments, the protein comprises an amino acid sequence with 93% to 100%, 94% to 100%, 96% to 100%, or 98% to 100% homology or identity to SEQ ID NO: 7. Each possibility represents a separate embodiment of the invention.

In some embodiments, the protein comprises or consists of the amino acid sequence:

(SEQ ID NO: 8)
MASSINISKIREAQRAQGPASILAVGTANPSNYEIQADFPDYYFRVTKSE
HMADMKGTFQRMCDKSMIRKRHMLITEEFLKENPNLCEYMAPSLDTRQDV
VVVEVPKLGKEAATKAIKEWGQPKSKITHLIFCTTTGVDMPGADYQLTKL
LGLAPSVKRFMIYQQGCFAGGTVLRLAKDIAENNKGARVLAVCSEITAMS
FRGPNDTHVDSLVGQALFGDGAAAVIVGSDPDLTTERPLFEIISAAQTIL
PNSEGAIDGHVREVGLTIHILKDVPVLISKNIEKALTQAFSPLGISDWNS
IFWIVHPGGPAILDQVELKVGLKKEKMATSRHVLSEYGNMSSACVFFIMD
EMRKRSAKGGARTTGEGLDWGVLFGFGPGLTVETVVLHSLPTTM.

In some embodiments, the protein comprises an amino acid sequence with at least 88%, at least 92%, at least 95%, or at least 97% homology or identity to SEQ ID NO: 8, or any value and range therebetween. Each possibility represents a separate embodiment of the invention. In some embodiments, the protein comprises an amino acid sequence with 88% to 100%, 91% to 100%, 93% to 100%, or 95% to 100% homology or identity to SEQ ID NO: 8. Each possibility represents a separate embodiment of the invention.

The terms “homology” or “identity”, as used interchangeably herein, refer to sequence identity between two amino acid sequences or two nucleic acid sequences, with identity being a stricter comparison. The phrases “percent identity or homology” and “% identity or homology” refer to the percentage of sequence identity found in a comparison of two or more amino acid sequences or nucleic acid sequences. Two or more sequences can be anywhere from 0-100% identical, or any value there between. Identity can be determined by comparing a position in each sequence that can be aligned for purposes of comparison to a reference sequence. When a position in the compared sequence is occupied by the same nucleotide base or amino acid, then the molecules are identical at that position. A degree of identity of amino acid sequences is a function of the number of identical amino acids at positions shared by the amino acid sequences. A degree of identity between nucleic acid sequences is a function of the number of identical or matching nucleotides at positions shared by the nucleic acid sequences. A degree of homology of amino acid sequences is a function of the number of amino acids at positions shared by the polypeptide sequences.

The following is a non-limiting example for calculating homology or sequence identity between two sequences (the terms are used interchangeably herein). The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frame shift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences.

In some embodiments, % homology or identity as described herein are calculated or determined using the basic local alignment search tool (BLAST). In some embodiments, % homology or identity as described herein are calculated or determined using Blossum 62 scoring matrix.

In some embodiments, the protein comprises or is characterized by polyketide synthesizing activity, as described herein. In some embodiments, the protein is characterized by having an activity of polymerizing a diketide substrate into a polyketide.

In some embodiments, a diketide substrate is obtained by coupling of an acyl CoA starting unit.

In some embodiments, an acyl CoA starting unit is selected form: acetyl COA, butyryl CoA, hexanoyl CoA, octanoyl CoA, cinnamoyl CoA, coumaroyl CoA, or any combination thereof.

In some embodiments, an acyl CoA is or comprises hexanoyl CoA, cinnamoyl CoA, or both.

In some embodiments, an acyl CoA is hexanoyl CoA.

In some embodiments, a polyketide comprises a tetraketide. In some embodiments, a polyketide comprises a linear polyketide. In some embodiments, a polyketide comprises a linear tetraketide.

According to some embodiments, there is provided a transgenic cell comprising: (a) the polynucleotide disclosed herein; (b) the artificial nucleic acid molecule disclosed herein; (c) the plasmid or agrobacterium disclosed herein; (d) the protein disclosed herein; or any combination thereof.

As used herein, the term “transgenic cell” refers to any cell that has undergone human manipulation on the genomic or gene level. In some embodiments, the transgenic cell has had exogenous polynucleotide, such as an isolated DNA molecule as disclosed herein, introduced into it. In some embodiments, a transgenic cell comprises a cell that has an artificial vector introduced into it. In some embodiments, a transgenic cell is a cell which has undergone genome mutation or modification. In some embodiments, a transgenic cell is a cell that has undergone CRISPR genome editing. In some embodiments, a transgenic cell is a cell that has undergone targeted mutation of at least one base pair of its genome. In some embodiments, the exogenous polynucleotide (e.g., the isolated DNA molecule disclosed herein) or vector is stably integrated into the cell. In some embodiments, the transgenic cell expresses a polynucleotide of the invention. In some embodiments, the transgenic cell expresses a vector of the invention. In some embodiments, the transgenic cell expresses a protein of the invention. In some embodiments, the transgenic cell, is a cell that is devoid of a polynucleotide of the invention that has been transformed or genetically modified to include the polynucleotide of the invention. In some embodiments, CRISPR technology is used to modify the genome of the cell, as described herein.

In some embodiments, the cell is a unicellular organism, a cell of a multicellular organism, and a cell in a culture.

In some embodiments, a unicellular organism comprises a fungus or a bacterium.

In some embodiments, the fungus is a yeast cell.

In some embodiments, the cell is an insect cell. In some embodiments, the cell comprises an insect cell line.

Types of insect cell lines suitable for transformation and/or heterologous expression are common and would be apparent to one of ordinary skill in the art. Non-limiting examples of such insect cell lines include, but are not limited to, Sf-9 cells, SR+Schneider cells, S2 cells, and others.

According to some embodiments, there is provided an extract derived from a transgenic cell disclosed herein, or any fraction thereof.

In some embodiments, the extract comprises the polynucleotide of the invention, an isolated DNA molecule as disclosed herein, an isolated protein as disclosed herein, or any combination thereof.

According to some embodiments, there is provided a homogenate, lysate, extract, derived from a transgenic cell disclosed herein, any combination thereof, or any fraction thereof.

Methods and/or means for extracting, lysing, homogenizing, fractionating, or any combination thereof, a cell or a culture of same, are common and would be apparent to one of ordinary skill in the art of cell biology and biochemistry. Non-limiting examples include, but are not limited to, pressure lysis (e.g., such as using a French press), enzymatic lysis, soluble-insoluble phase separation (such for obtaining a supernatant and a pellet), detergent-based lysis, solvent (e.g., polar or nonpolar solvent), liquid chromatography mass spectrometry, or others.

According to some embodiments, there is provided a transgenic plant, a transgenic plant tissue or a plant part. In some embodiments, there is provided a transgenic plant, or any portion, seed, tissue or organ thereof, comprising at least one transgenic plant cell of the invention. In some embodiments, the transgenic plant, transgenic plant tissue or plant part, comprises: (a) the polynucleotide disclosed herein; (b) the artificial disclosed herein; (c) the plasmid or agrobacterium disclosed herein; (d) the isolated protein of the invention; (e) the transgenic cell disclosed herein; or any combination thereof.

In some embodiments, the transgenic plant, transgenic plant tissue, or plant part consists of transgenic plant cells of the invention. In some embodiments, the transgenic plant, transgenic plant tissue, or plant part comprises at least: 20%, 25%, 30%, 35%, 40%, 45%, 50%, 55%, 60%, 65%, 70%, 75%, 80%, 85%, 90%, 95%, 97%, or 99% transgenic cells of the invention, or any value and range therebetween. Each possibility represents a separate embodiment of the invention. In some embodiments, the transgenic plant, transgenic plant tissue, or plant part comprises 20%-50%, 20%-60%, 20%-70%, 20%-80%, 20%-90%, or 20%-100% transgenic cells of the invention. Each possibility represents a separate embodiment of the invention.

In some embodiments, the transgenic plant, transgenic plant tissue, or plant part is or derived from a Cannabis sativa plant. In some embodiments, the transgenic plant is a C. sativa plant.

In some embodiments, the transgenic plant, transgenic plant tissue, or plant part is or derived from hemp. In some embodiments, C. sativa comprises or is hemp.

According to some embodiments, there is provided a composition comprising any one of the herein disclosed: (a) polynucleotide of the invention (for example, an isolated DNA molecule); (b) artificial vector; (c) plasmid or agrobacterium; (d) isolated protein of the invention; (e) transgenic cell; (f) extract; (g) transgenic plant tissue or plant part; and (h) any combination of (a) to (g), and an acceptable carrier.

As used herein, the term “carrier”, “excipient”, or “adjuvant” refers to any component of a composition, e.g., pharmaceutical or nutraceutical, that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier (e.g. carbomer, hydroxypropyl cellulose, sodium lauryl sulfate) as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers, and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers, and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

### Methods of Synthesis

According to some embodiments, there is provided a method for synthesizing a polyketide.

According to some embodiments, the method comprises the steps: (a) providing a cell comprising an artificial vector comprising a nucleic acid sequence having at least 82%, at least 85%, at least 89%, at least 92%, at least 95%, or at least 99% homology or identity to any one of SEQ ID Nos: 1-4, or any combination thereof, or any value and range therebetween; and (b) culturing the cell from step (a) such that a protein encoded by the artificial vector is expressed, thereby synthesizing a polyketide. Each possibility represents a separate embodiment of the invention.

According to some embodiments, the method comprises contacting a diketide substrate with an effective amount of a protein comprising an amino acid sequence with at least 88%, at least 91%, at least 95%, or at least 97% homology or identity to any one of SEQ ID Nos: 5-8, or any value and range therebetween, thereby synthesizing a polyketide. Each possibility represents a separate embodiment of the invention.

According to some embodiments, there is provided a method for obtaining an extract from a transgenic cell or a transfected cell.

In some embodiments, the method comprises culturing a transgenic cell or a transfected cell in a medium and extracting the transgenic cell or the transfected cell.

In some embodiments, the method comprises the steps: (a) culturing a transgenic cell or a transfected cell in a medium; and (b) extracting the transgenic cell or the transfected cell, thereby obtaining an extract from the transgenic cell or the transfected cell.

In some embodiments, the transgenic cell or the transfected cell comprises an artificial vector comprising a nucleic acid sequence having at least 82%, at least 87%, at least 91%, at least 93%, at least 95%, at least 97%, at least 99%, or 100% homology or identity to any one of SEQ ID Nos: 1-4, or any combination thereof, or any value and range therebetween. Each possibility represents a separate embodiment of the invention.

In some embodiments, the transgenic cell or the transfected cell comprises the polynucleotide of the invention or a plurality thereof, as disclosed herein.

In some embodiments, the transgenic cell or the transfected cell comprises the artificial nucleic acid molecule or vector as disclosed herein.

In some embodiments, the cell is a transgenic cell, or a cell transfected with a polynucleotide as disclosed herein.

In some embodiments, the culturing comprises supplementing the cell with an effective amount of a diketide substrate, an acyl CoA starting unit, or both. In some embodiments, the supplementing is via the growth or culture medium wherein the cell is cultured.

In some embodiments, the diketide substrate is obtained by coupling of an acyl CoA starting unit. In some embodiments, the diketide substrate is a substrate of a protein encoded by a polynucleotide as disclosed herein. In some embodiments, the diketide substrate is a substrate of a protein as disclosed herein. In some embodiments, the diketide is a substrate of a PKS enzyme as disclosed herein (e.g., a protein encoded by the polynucleotide of the invention and/or the protein of the invention).

In some embodiments, the acyl CoA is selected form: acetyl COA, butyryl CoA, hexanoyl CoA, octanoyl CoA, cinnamoyl CoA, coumaroyl CoA, or any combination thereof.

In some embodiments, the acyl CoA is or comprises hexanoyl CoA.

In some embodiments, the method further comprises a step preceding step (a), comprising introducing or transfecting the cell with the artificial nucleic acid molecule or vector, disclosed herein.

Method for introducing or transfecting a cell with an artificial nucleic acid molecule or vector are common and would be apparent to one of ordinary skill in the art.

In some embodiments, introducing or transfecting comprises transferring an artificial nucleic acid molecule or vector comprising the polynucleotide disclosed herein into a cell; or modifying the genome of a cell to include the polynucleotide disclosed herein. In some embodiments, the transferring comprises transfection. In some embodiments, the transferring comprises transformation. In some embodiments, the transferring comprises lipofection. In some embodiments, the transferring comprises nucleofection. In some embodiments, the transferring comprises viral infection.

As used herein, the terms “transfecting” and “introducing” are interchangeable.

In some embodiments, the contacting is in a cell-free system.

Types of suitable cell-free systems for synthesizing a polyketide utilizing any one of: the polynucleotide of the invention or a plurality thereof, as disclosed herein, and the protein of the invention, or a plurality thereof, would be apparent to one of ordinary skill in the art.

In some embodiments, the method further comprises a step preceding step (b), comprising separating the cultured transgenic cell or the cultured transfected cell from the medium.

Method for separating cell from a medium are common and may include, but not limited to, centrifugation, ultracentrifugation, or other, as would be apparent to one of ordinary skill in the art.

According to some embodiments, there is provided an extract of a transgenic cell or a transfected cell obtained according to the herein disclosed method.

According to some embodiments, there is provided a medium or a portion thereof separated from a cultured transgenic cell or a cultured transfected cell, obtained according to the herein disclosed method.

According to some embodiments, there is provided a composition comprising: (a) the extract disclosed herein; (b) the medium disclosed herein or a portion thereof; or (c) any combination of (a) and (b), and an acceptable carrier, as described herein.

In some embodiments, a portion comprises a fraction or a plurality thereof.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the term “about” when combined with a value refers to plus and minus 10% of the reference value. For example, a length of about 1,000 nanometers (nm) refers to a length of 1,000 nm±100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Itis appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization-A Laboratory Course Manual” CSHL Press (1996); all of which are incorporated by reference. Other general references are provided throughout this document.

### Materials and Methods

### Chemicals and Reagents

Unless otherwise stated, all the analytical compounds were ≥95% pure. Malonyl-CoA (≥90%), hexanoyl-CoA (≥85%) and olivetol were purchased from Sigma-Aldrich (Rehovot, Israel). OA (>90%) was purchased from Cayman Chemical (Ann Arbor, MI, USA). Hexanoylphloroglucinol (THPH) was purchased from Wuhan ChemFaces Biochemical Co Ltd. (Hubei, China).

### Trichome Isolation

Young leaves were harvested and soaked in ice-cold, distilled water and then abraded using a BeadBeater machine (Biospec Products, Bartlesville, OK). The polycarbonate chamber was filled with 15 g of plant material, and with half the volume with glass beads (0.5 mm diameter), XAD-4 resin (1 g/g plant material), and ethanol 80% to full volume. Leaves were beated by 2-4 pulses of operation of 1 min each. This procedure was carried out at 4 °C, and after each pulse the chamber was allowed to cool on ice. Following abrasion, the contents of the chamber were first filtered through a kitchen mesh strainer and then through a 100 μm nylon mesh to remove the plant material, glass beads, and XAD-4 resin. The residual plant material and beads were scraped from the mesh and rinsed twice with additional ethanol 80% that was also passed through the 100 μm mesh. The presence of enriched glandular trichome secretory cells was checked by visualization in an inverted optical microscope.

Genome Sequencing and Assembly of H. umbraculigerum

The genome size of H. umbraculigerum was estimated by flow cytometry. Briefly, nuclei were isolated by chopping young leaf tissue of Helichrysum and tomato (used as known reference) in isolation buffer. The samples were stained with propidium iodide, and at least 10,000 nuclei were analyzed in a flow cytometer, and the ratio of G1 peak means between both samples was calculated. High molecular weight DNA was extracted from young frozen leaves and sent for sequencing in the Genome Center of UC Davis. The DNA quality was checked by TapeStation traces and a Qubit fluorimeter (Thermo Fisher). Sequencing was done in a Pacbio Sequel II platform, and a ~12-kilobase DNA SMRT bell library was prepared according to the manufacturer's protocol. Three different SMRT 8M cells were used, yielding 57.8 Gb of HiFi data (~44× haploid coverage). In addition to Pacbio HiFi data, 200 M reads of PE 2×150 Illumina Hi-C data were obtained by Phase Genomics. Hifiasm software was used to integrate both Pacbio HiFi and HiC data to produce chromosome-scale and haplotype-resolved assemblies.

Further scaffolding of the primary assembly was performed using the Hi-C data and the SALSA software. Ragtag was used for a final round of ordering using the primary assembly as reference to reach syntenic scaffolds for each haplotype. Visualizations of Hi-C data were performed with Juicer and whole-genome alignments with the pafr package (https://dwinter.github.io/pafr/). Finally, the assembly was softmasked for repetitive elements using EDTA.

RNA Sequencing and Genome Annotation of H. umbraculigerum

RNA was extracted from seven different tissues: young leaves, old leaves, florets and receptacles of flowers, stems, roots and trichomes. RNA integrity was checked using a TapeStation instrument. Paired-end Illumina libraries were prepared for five of the tissues and sequenced on Illumina HiSeq 3000 instrument (PE 2×150, ~40 M reads per sample). Random sequencing errors were corrected using Rcorrector18, and uncorrectable reads were removed. Adaptor and quality trimming were performed using TrimGalore! with the following parameters:—length 36 b-5—stringency 1-e 0.1 (github.com/FelixKrueger/TrimGalore). Ribosomal RNA was filtered by discarding reads mapping to SILVA_132_LSURef and SILVA 138 SSURef non-redundant databases using bowtie2—very-sensitive-local mode. Fastq quality checks on each of the steps were performed using MultiQC. The remaining reads were pooled and used for genome-guided de novo transcriptome assembly using Trinity. The Iso-Seq data were obtained from four of the tissues and processed using isoseq3 and cDNA Cupcake ToFU pipelines (github.com/Magdoll/cDNA_Cupcake). Fused and unspliced transcripts were removed, and only polyA positive transcripts were kept for a unique set of high-quality isoforms. Iso-Seq and Trinity transcripts were aligned to the assembly using minimap2 and the BAM files were used in the PASA pipeline to generate RNA-based gene model structures. In addition, the novo gene structures were obtained using the software braker2 and the mentioned BAM files as extrinsic training evidence. Finally, ab initio and RNA-based gene models were combined using EvidenceModeler and a final round of PASA pipeline. Gene functional annotation was performed for the predicted mature transcripts using TransDecoder (github.com/TransDecoder/TransDecoder), which considers HMMER hits against PFAM and BLASTP hits against UniProt databases for similarity retention criteria. Further annotation of protein-coding transcripts was performed by BLASTP searches against curated plant protein databases and GO and KEGG terms were obtained with Triannotate.

UMI-based 3′ RNAseq of three replicates of the seven tissues was obtained similarly as described. Adaptor and quality trimming were performed using TrimGalore! in two steps, including PolyA trimming mode. Reads were mapped to the genome using STAR, UMI-deduplicated using umitools, and counts were obtained with featureCounts. Normalization was performed with the varianceStabilizingTransformation algorithm of DESeq2, and the CEMItools package was used for coexpression analysis (dissimilarity threshold of 0.6, pvalue of 0.1). Genes in modules with expression profiles in concordance with the presence of the metabolites of interest were analyzed. Candidate genes were selected based on functional annotations, and blast hits with known enzymes.

### PKS Candidate Gene Cloning

For heterologous expression in SoluBL21 E. coli, coding sequences of HuPKS candidates from H. umbraculigerum were amplified. Due to the high sequence similarity of the coding sequences, HuPKS2-4 were synthesized by the company Twist Biosciences. Moreover, PKC (i.e., CsOAC) known to be involved in olivetolic acid biosynthesis in Cannabis sativa was also amplified. The PCR products were purified from agarose gel and ligated into the pOPINF vector (digested with HindIII and KpnI) using the ClonExpress II one step cloning kit (Vazyme, Germany). Infusion reactions were transformed into competent E. coli Stellar cells (Clontech Takara). Recombinant colonies were selected on LB agar plates supplemented with ampicillin (100 μg/mL). Positive clones were confirmed by Sanger sequencing.

### Protein Expression and Purification

Genes cloned into pOPINF vectors were transformed into SoluBL21 E. coli strain for protein expression. Single colonies grown in LB agar media were picked and grown overnight in 10 mL LB media at 37 °C. The next day, 1 mL of the overnight cultures were used to inoculate 100 mL of LB media. The fresh cultures were grown at 37 °C until OD₆₀₀ of 0.6-0.8 was reached, followed by induction with 600 μM IPTG and overnight incubation at 16 °C. For purification, the cells were harvested by centrifugation (10 min at 3200×g) and lysed by sonication in 50 mM Tris-HCl pH 8, 0.5 mM phenylmethylsulfonyl fluoride (PMSF, Sigma Aldrich) solution in isopropanol, 10% glycerol and protease inhibitor cocktail (Sigma Aldrich), and 0.1 mg ml-1 lysozyme (Sigma Aldrich). Purification of proteins was performed on Ni-NTA agarose beads (Adar Biotech). The proteins were eluted with 200 mM imidazole (Fluka) in buffer containing 50 mM NaH₂PO₄, pH 8.0, and 0.5 M NaCl. Dialysis and buffer exchange was performed using 20 mM HEPES pH 7.2 in centrifugal concentrators with size exclusion of 3 or 10 KDa depending on the protein size. The recombinant enzymes were verified by SDS-PAGE analysis, and the protein concentrations were measured with Pierce™ 660 nm protein assay reagent (Thermo Scientific).

### HuPKS Enzyme Assays for Olivetolic Acid Production

Individual and coupled HuPKS and CsOAC assays were carried out as described by Gagne et al., (2012) with some modifications. Enzyme assays were performed in 50 μL with 20 mM HEPES at pH 7.2, 5 mM DTT, 1.8 mM malonyl CoA and 0.6 mM of hexanoyl-CoA. HuPKSs (5 μg) and CsOAC (10 μg), were added either individually or in combination. Reaction mixtures were incubated at 30 °C for 3 h. Reactions were stopped by extraction with 100 μL MeOH, vortexing and centrifugation at 15 000 g for 10 min. The supernatant was filtered and analyzed with ultra-high performance liquid chromatography connected to quadrupole time-of-flight (UPLC-qTOF) or triple-Quad systems. The chromatographic separation was performed on a 100 mm×2.1 mm i.d. (internal diameter), 1.7 μm UPLC BEH C18 column (Waters Acquity). The mobile phase consisted of 0.1% formic acid in acetonitrile:water (5:95, v/v; phase A) and 0.1% formic acid in acetonitrile (phase B). The flow rate was 0.3 ml min⁻¹, and the column temperature was kept at 35 °C. Compounds were analyzed using an 11 min multistep gradient method: initial conditions were 10% B raised to 70% until 6 min, raised to 100% B until 6.2 min, held at 100% B until 8 min, decreased to 10% B until 8.5 min, and held at 10% B until 11 min for re-equilibration of the system. Electrospray ionization (ESI) was used in negative or positive ionization with an m/z range of 50-1,000 Da. Masses of the eluted compounds were detected with the following settings: source temperature 140 °C, desolvation temperature 450 °C, and desolvation gas flow 800 l h⁻¹; capillary 1.0 kV in negative mode and 1.5 kV in positive mode. Argon was used as the collision gas. MS/MS experiments were performed in negative or positive ionization modes according to the specific masses of the deprotonated or protonated compounds. The following settings were used: cone voltage of 30 eV; collision energy ramp of 15-50 eV in negative and 10-45 eV in positive modes.

Triple-Quad analyses were performed on a TQ-S system in MRM mode using a similar column and gradient as previously described. The instrument was operated in both positive and negative modes with a capillary voltage of 3.5 or 1.5 kV, respectively, and a cone voltage of 40 or 20 V, respectively. Two different transitions were used for analysis of: Olivetolic acid (223.1>179.1, 15.0 V; 223.1>137.1, 20.0 V); PDAL (181.2>137.1, 10.0 V; 181.2>97.1, 20.0 V); HTAL (223.1>179.1, 10.0 V; 223.1>125.1, 10.0 V); THPH (223.1>179.1, 20.0 V; 223.1>81.0, 25.0 V); in negative mode; and olivetol (181.1>111.0, 10.0 V; 181.1>71.2, 10.0 V) in positive mode.

### Example 1

### Polyketide Synthase (PKS) Enzymes in H. umbraculigerum

OA is the first key intermediate in the cannabinoid biosynthetic pathway in Cannabis sativa (Cannabis). The biosynthesis of OA from hexanoyl CoA involves two enzymatic reactions catalyzed namely by a type III PKS, olivetol synthase (CsOLS). This enzyme first converts hexanoyl CoA to a tetraketide intermediate, that is further modified to OA by a polyketide cyclase (PKC)-type olivetolic acid cyclase (CsOAC) (Taura et al., 2009; Gagne et al., 2012). To identify genes in H. umbraculigerum associated with CsOLS-like activity, the inventors searched for candidate type III PKS genes in H. umbraculigerum genome. Four PKS-like genes, namely HuPKS1 (SEQ ID NO: 1), HuPKS2 (SEQ ID NO: 2), HuPKS3 (SEQ ID NO: 3) and HuPKS4 (SEQ ID NO: 4) enzymes were selected for further characterization based on their differential expression profile in leaves and in trichomes compared to other tissues.

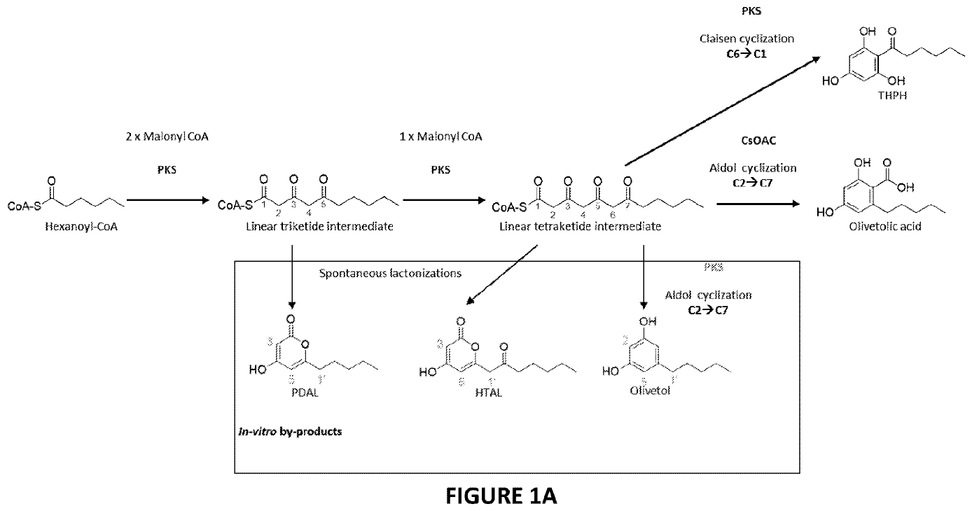

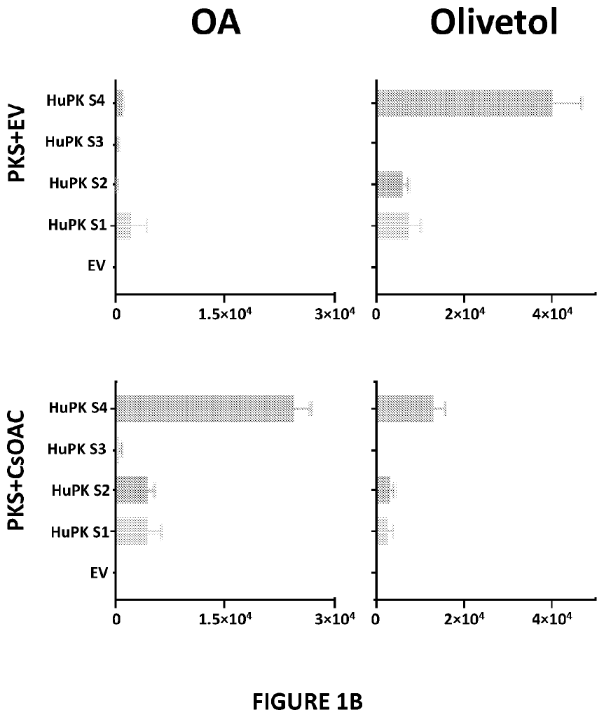

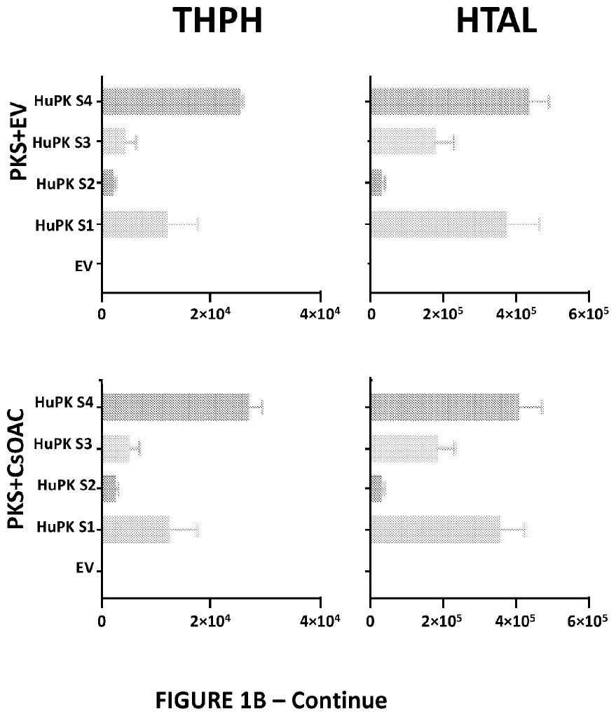

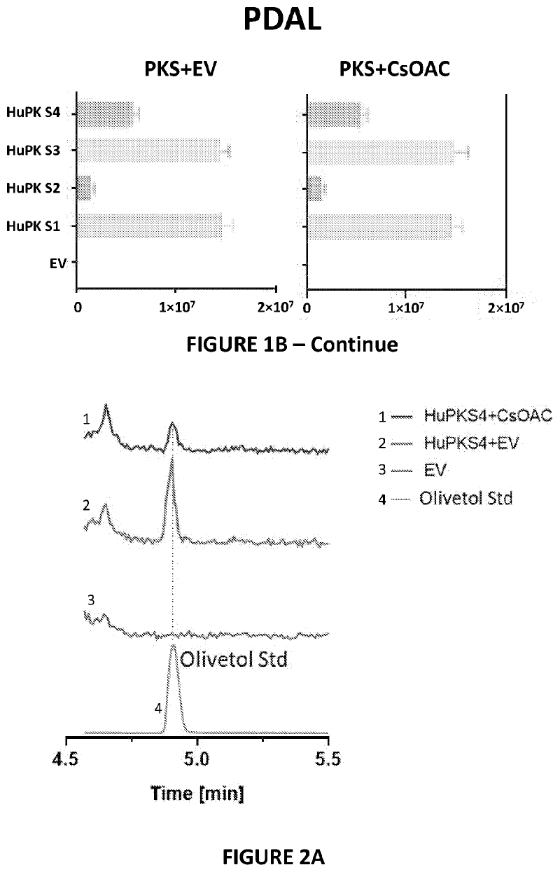

Next, the inventors expressed HuPKS1-4 enzymes and CsOAC enzymes and tested using hexanoyl-CoA and malonyl-CoA their ability to form OA individually or in coupled in-vitro assays. It is known that in in-vitro assays derailment of the unstable intermediates occurs producing additional by-products not naturally identified in plant extracts [olivetol, pentyl acyl diacetic acid lactone (PDAL) and hexanoyl acyl triacetic acid lactone (HTAL)]. PDAL and HTAL are produced by spontaneous lactonization of the tri- and tetra-ketide unstable intermediates, whereas CsOLS produces olivetol in the absence of CsOAC in an Aldol decarboxylation cyclization reaction resembling the production of resveratrol by a stilbene synthase (FIG. 1A).

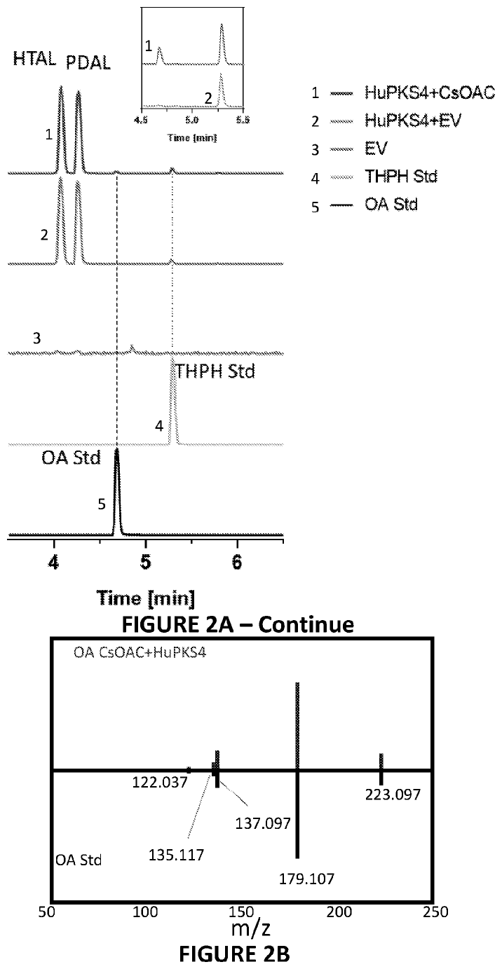

In the current study, the inventors show that similarly to CsOLS, in the absence of CsOAC, all the HuPKSs produced the PDAL and HTAL by-products; while HuPKS1, HuPKS2 and HuPKS4 produced also olivetol (FIG. 1B). Verification of OA and olivetol production using LC-HRMS appears in FIG. 2. When the reactions were performed coupled to CsOAC, olivetol decreased and OA increased, especially for HuPKS2 and HuPKS4 (FIG. 1B). Notably, all the HuPKSs produced also the phloroglucinoid precursor hexanoylphloroglucinol (THPH) regardless of CsOAC (FIG. 1B), meaning that the same HuPKS enzyme can produce both the Aldol and Claisen cyclization reactions (FIG. 1A).

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

1. An isolated DNA molecule comprising a nucleic acid sequence having at least 83% homology to any one of SEQ ID Nos: 1-4, or any combination thereof.

2. The isolated DNA molecule of claim 1, wherein said nucleic acid sequence having at least 83% homology to any one of SEQ ID Nos: 1-4 is 1,000 to 1,400 nucleotides long, and optionally wherein said nucleic acid sequence encodes a protein being a polyketide synthase.

3. (canceled)

4. An artificial nucleic acid molecule, a plasmid, or an agrobacterium comprising the isolated DNA molecule of claim 1.

5. (canceled)

6. An isolated protein encoded by the isolated DNA molecule of claim 1.

7. The isolated protein of claim 6, comprising or consisting of an amino acid sequence with at least 93% homology to any one of SEQ ID Nos: 5-8.

8. (canceled)

9. The isolated protein of claim 7, wherein (a) said isolated protein being characterized by having an activity of polymerizing a diketide substrate into a polyketide, optionally wherein said diketide substrate is obtained by coupling of an acyl CoA starting unit, and optionally wherein: (i) said acyl CoA starting unit is selected form the group consisting of: acetyl COA, butyryl CoA, hexanoyl CoA, octanoyl CoA, cinnamoyl CoA, coumaroyl CoA, and any combination thereof; (ii) said acyl CoA is hexanoyl CoA, cinnamoyl CoA, or both; or (iii) both (i) and (ii); (b) said polyketide comprises a tetraketide; or (c) both (a) and (b).

10.-13. (canceled)

14. A transgenic cell comprising the isolated DNA molecule of claim 1.

15. The transgenic cell of claim 14, being any one of: a unicellular organism, a cell of a multicellular organism, and a cell in a culture, optionally wherein said unicellular organism comprises a fungus or a bacterium, and optionally wherein said fungus is a yeast cell.

16.-17. (canceled)

18. An extract derived from the transgenic cell of claim 14, or any fraction thereof, and optionally wherein said extract comprises said nucleic acid sequence, an isolated protein encoded therefrom, or both.

19. (canceled)

20. A transgenic plant, a transgenic plant tissue or a plant part, comprising the isolated DNA molecule of claim 1, and optionally wherein said transgenic plant is a Cannabis sativa plant.

21. (canceled)

22. A composition comprising the isolated DNA molecule of claim 1, and an acceptable carrier.

23. A method for synthesizing a polyketide comprising the steps:
a. providing a cell comprising an artificial vector comprising a nucleic acid sequence having at least 83% homology to any one of: SEQ ID Nos: 1-4; and
b. culturing said cell from step (a) such that a protein encoded by said artificial vector is expressed,
thereby synthesizing a polyketide.

24. The method of claim 23, wherein any one of: (i) said protein is characterized by having an activity of polymerizing a diketide substrate into a polyketide; (ii) said polyketide comprises a tetraketide, and optionally wherein said diketide substrate is obtained by coupling of an acyl CoA starting unit; (iii) said culturing comprises supplementing said cell with an effective amount of a diketide substrate, an acyl CoA starting unit, or both, and optionally wherein said acyl CoA starting unit is selected form the group consisting of: acetyl COA, butyryl CoA, hexanoyl CoA, octanoyl CoA, cinnamoyl CoA, coumaroyl CoA, and any combination thereof, said acyl CoA starting unit is hexanoyl CoA, cinnamoyl CoA, or both; or both; (iv) said artificial vector is an expression vector; (v) said cell is a prokaryote cell or a eukaryote cell; (vi) said cell is a transgenic cell or a cell transfected with an isolated DNA molecule comprising a nucleic acid sequence having at least 83% homology to any one of SEQ ID Nos: 1-4, or any combination thereof or an artificial vector comprising thereof; (vii) said method further comprises a step preceding step (a), comprising introducing or transfecting said cell with said artificial vector; and (viii) any combination of (i) to (vii).

25.-33. (canceled)

34. A method for synthesizing a polyketide comprising contacting a diketide substrate with an effective amount of protein comprising an amino acid sequence with at least 93% homology to any one of SEQ ID Nos: 5-8, thereby synthesizing a polyketide.

35. The method of claim 34, wherein: (i) said polyketide comprises a tetraketide; (ii) said contacting is in a cell-free system; or both (i) and (ii), optionally wherein said diketide substrate is obtained by coupling of an acyl CoA starting unit, and optionally wherein said acyl CoA starting unit is selected form the group consisting of: acetyl COA, butyryl CoA, hexanoyl CoA, octanoyl CoA, cinnamoyl CoA, coumaroyl CoA, and any combination thereof, said acyl CoA starting unit is hexanoyl CoA, cinnamoyl CoA, or both; or both.

36.-39. (canceled)

40. A method for obtaining an extract from a transgenic cell or a transfected cell comprising the steps:
a. culturing a transgenic cell or a transfected cell in a medium, wherein said transgenic cell or said transfected cell comprises the isolated DNA molecule of claim 1; and
b. extracting said transgenic cell or said transfected cell,
thereby obtaining an extract from the transgenic cell or the transfected cell.

41. The method of claim 40, further comprising a step preceding step (b), comprising separating said cultured transgenic cell or said cultured transfected cell from said medium.

42. An extract of a transgenic cell or a transfected cell obtained according to the method of claim 40.

43. A medium or a portion thereof separated from a cultured transgenic cell or a cultured transfected cell, obtained according to the method of claim 41.

44. A composition comprising the extract of claim 42, and an acceptable carrier.


SEQUENCE LISTING (8 sequences)

SEQ ID NO: 1 - DNA, 1197 bp, Helicrysum umbraculigerum
  source 1..1197: mol_type = other DNA
atggcatcctcaattaatatctccaagatcagagaggctcaacgagcacaaggtccagcctctattcttgctgtcggtaccgcgaatccgtctaattgcgtgtatcaagctgattatcctgattactactttcgaatcactaaaagtgaacacatggttgatctcaaacggaaattcaagcgcatgtgtgaccaatctatgataagaaagcggtacatgcaaattacggaggagtatctgaaagaaaaccccaacatttgtgaatacatggctccatcacttgacgcccgtcaagacgttgtagtcgtcgaagtcccaaaactcggtaaagaagccgcaacaaaagccatcaaagaatggggccaaccaaaatccaaaattacccatctcatcttttgtaccacgtccggtgtcgacatgcccggagcagattaccagctcaccaaactcctcggtctttgtccttcagtcaaacgctttatgatgtaccaacaaggttgttttgctggtggcacggttcttcgtctagctaaggacatcgctgagaacaataaaggtgctcgtgtacttgtcgtttgttccgagattacagctgtcatttttcgtggacccaacgacactcaccttgattcacttatcggtcaagcgttatttggggatggggcatcttcggttatcgtggggtctgacccagacttgacaaccgagcggccattgtttgaaatcatatcggctgcacaaacgattttaccggactctgaaggtgcgatagatggacacttgagggaagctgggttaacttttcatctacttaaagacgtaccgaggttgatttcgaagaatatagagaaagctttaacacaagcattttctcccctgggaattagtgactggaactctatcttttgggtcacgcaccctggtggtccagctatactggaccaagtggaactcaaacttggactcaaagaggagaagatgagaaccactagacatgttctcagtgaatatgggaacatgtctagtgcatgtgttttttttgtacttgatgaaatgagaaagagatcggctaaaggcggtgcgaggaccaccggagaagggttagattggggtgttctgtttgggtttggtccgggtttaacggttgagactgtggtccttcatagtctcccaactactatgtcgattgcgacttaa

SEQ ID NO: 2 - DNA, 1197 bp, Helicrysum umbraculigerum
  source 1..1197: mol_type = other DNA
atggcatcctcaattaatatctccaagatcagagaggctcaacgagcacaaggtccagcctctattcttgctgtcggtactgcgaatccgtctaattgtgtgtatcaagctgattatcctgattactactttcgaatcactaaaagtgaacacatggttgatttgaaagagaaattccagcgcatgtgtgacaaatctatgataagaaagcggcacattcacattacggaggagtttttgaaagaaaacccaaacctttgtgaatacatggctccatcacttgacacccgtcaagacgttgtagtcgtcgaagtcccaaaactcggtaaagaagccgcaacaaaagccatcaaagaatggggccaaccaaaatccaaaattacccatctcatcttttgtaccacgtccggtgtcgacatgcccggagcagattaccagctcaccaaactcctcggtctccatccttcagtcaaacgctttatgatgtaccaacaaggttgttttgctggtggcacggttcttcgtctagctaaggacctcgctgagaacaataaaggtgctcgtgtacttgccgtttgttccgagattacagctgtcacgtttcgtggacccaacgacactcacattgattcacttgtcggtcaagcattatttggggacggggcagctgcggttatcgtggggtctgatcctgacttgacaactgagcggccgttgtttgaaatcatatcggctgcacaaacgattttaccgaactctgaaggtgcgatagatggacatgtgagggaagttggggtaactattcatatacttaaagacgtcccggtgttgatttcgaagaatatagagaaagctttaacacaagcattttctcccttaggaattagtgactggaactcgatcttttgggtcgtacaccctggtggtccagctatactggaccaagtggaactcaaacttggactcaaagaggagaaaatgagaaccactagacatgttctcagtgaatatgggaacatgtctagtgcatgtgttttttttgtacttgatgaaatgagaaagagatcggctaaaggcggtgcgaggaccaccggagaagggttagattggggtgttctgtttgggtttggtccaggtttaacggttgagacggtggtccttcatagtctcccaactactatgtcgattgcaacttaa

SEQ ID NO: 3 - DNA, 1197 bp, Helicrysum umbraculigerum
  source 1..1197: mol_type = other DNA
atggcatcctcaattaatatctccaagatcagagaggctcaacgagcacaaggtccagcctctattcttgctgtcggtaccgcgaatccgtctaattgcgtgtatcaagctgattatcctaattactactttcgaatcactaaaagtgaacacatggttgatctcaaacggaaattcaagcgcatgtgtgaccaatctatgataagaaagcggtacatgcaaattacggaggagtatctgaaagaaaaccccaacatttgtgaatacatggctccatcacttgacgcccgtcaagacgttgtagtcgtcgaagtcccaaaactcggtaaagaagccgcaacaaaagccatcaaagaatggggccaaccaaaatccaaaattacccatctcatcttttgtaccacgtccggtgtcgacatgcccggagcagattaccagctcaccaaactcctcggtctctgtccttcagtcaaacgctttatgatgtaccaacaaggttgttttgctggtggcacggttcttcgtctagctaaggacatcgctgagaacaataaaggtgctcgtgtacttgtcgtttgttccgagattacagctgtcatttttcgtggacccaacgacactcaccttgattcacttatcggtcaagcgttatttggggatggggcatcttcggttatcgtggggtctgacccagacttgacaaccgagcggccattgtttgaaatcatatcggctgcacaaacgattttaccggactctgaaggtgcgatagatggacacttgagggaagctgggttaacttttcatctacttaaagacgtaccggggttgatttcgaagaatatagagaaagctttaacacaagcattttctcccttgggaattagtgactggaactctatcttttgggtcacgcaccctggtggtccagctatactggaccaagtggaactcaaacttggactcaaagaggagaagatgagagcctctagacatgttctcagtgaatacgggaacatgtctagtgcatgtgttttttttatacttgatgaaatgagaaagaaatcggatgaagatggtgcgccgaccactggagaagggttagattggggtgttctgtttgggtttggtccgggtttaacggttgagacggtggtccttcatagtctcccaactactatgtcgattgcgacttaa

SEQ ID NO: 4 - DNA, 1185 bp, Helicrysum umbraculigerum
  source 1..1185: mol_type = other DNA
atggcatcctcaattaatatctctaagatcagagaggctcaacgagcacaaggtccagcctctattcttgctgtcggtactgcgaatccatctaattatgagattcaagctgattttcctgattactactttcgagtcactaaaagtgaacacatggctgatatgaaagggacattccagcgcatgtgtgacaaatctatgataagaaagcggcacatgctcattacggaggagtttttgaaagaaaacccaaacctttgtgaatacatggctccatcacttgacacccgtcaagacgttgtagtcgtcgaagtcccaaaactcggtaaagaagccgcaacaaaagccatcaaagaatggggccaaccaaaatccaaaattacccatctcatcttttgtactacaactggtgtcgacatgcctggagccgattaccagctcaccaagctcctcggcctcgctccttcagtcaaacgctttatgatataccaacaaggttgttttgctggtggcacggttcttcgtcttgctaaagacatagctgagaacaataaaggtgctcgtgtacttgccgtatgttcagagattacagctatgtcgtttcgtgggcccaatgacactcacgttgattcacttgtcggtcaagcattatttggggacggggcagctgcagttatcgtggggtctgatcctgacttgacaaccgagcggccgttgtttgaaatcatatcggctgcacaaacgattttaccaaactctgaaggtgcgatagatggacatgtgagggaagttggtttaactattcatatacttaaagacgtcccggtgttgatatcgaagaatatagagaaagctttgacacaagcattttctcccttaggaattagtgactggaactcgatcttttggatcgtacaccctggtggtccagctatactggaccaagtggaactcaaagttggactcaaaaaggagaaaatggcaaccagtagacatgttctaagtgaatacgggaacatgtctagtgcatgtgttttttttataatggatgaaatgagaaagagatcggctaaaggcggtgcgaggaccaccggagaagggttagattggggtgttttgtttgggtttggtccaggtttaacggttgagacggtggtccttcatagtctcccaactacaatgtag

SEQ ID NO: 5 - protein, 398 aa, Helicrysum umbraculigerum
MASSINISKIREAQRAQGPASILAVGTANPSNCVYQADYPDYYFRITKSEHMVDLKRKFKRMCDQSMIRKRYMQITEEYLKENPNICEYMAPSLDARQDVVVVEVPKLGKEAATKAIKEWGQPKSKITHLIFCTTSGVDMPGADYQLTKLLGLCPSVKRFMMYQQGCFAGGTVLRLAKDIAENNKGARVLVVCSEITAVIFRGPNDTHLDSLIGQALFGDGASSVIVGSDPDLTTERPLFEIISAAQTILPDSEGAIDGHLREAGLTFHLLKDVPRLISKNIEKALTQAFSPLGISDWNSIFWVTHPGGPAILDQVELKLGLKEEKMRTTRHVLSEYGNMSSACVFFVLDEMRKRSAKGGARTTGEGLDWGVLFGFGPGLTVETVVLHSLPTTMSIAT

SEQ ID NO: 6 - protein, 398 aa, Helicrysum umbraculigerum
MASSINISKIREAQRAQGPASILAVGTANPSNCVYQADYPDYYFRITKSEHMVDLKEKFQRMCDKSMIRKRHIHITEEFLKENPNLCEYMAPSLDTRQDVVVVEVPKLGKEAATKAIKEWGQPKSKITHLIFCTTSGVDMPGADYQLTKLLGLHPSVKRFMMYQQGCFAGGTVLRLAKDLAENNKGARVLAVCSEITAVTFRGPNDTHIDSLVGQALFGDGAAAVIVGSDPDLTTERPLFEIISAAQTILPNSEGAIDGHVREVGVTIHILKDVPVLISKNIEKALTQAFSPLGISDWNSIFWVVHPGGPAILDQVELKLGLKEEKMRTTRHVLSEYGNMSSACVFFVLDEMRKRSAKGGARTTGEGLDWGVLFGFGPGLTVETVVLHSLPTTMSIAT

SEQ ID NO: 7 - protein, 398 aa, Helicrysum umbraculigerum
MASSINISKIREAQRAQGPASILAVGTANPSNCVYQADYPNYYFRITKSEHMVDLKRKFKRMCDQSMIRKRYMQITEEYLKENPNICEYMAPSLDARQDVVVVEVPKLGKEAATKAIKEWGQPKSKITHLIFCTTSGVDMPGADYQLTKLLGLCPSVKRFMMYQQGCFAGGTVLRLAKDIAENNKGARVLVVCSEITAVIFRGPNDTHLDSLIGQALFGDGASSVIVGSDPDLTTERPLFEIISAAQTILPDSEGAIDGHLREAGLTFHLLKDVPGLISKNIEKALTQAFSPLGISDWNSIFWVTHPGGPAILDQVELKLGLKEEKMRASRHVLSEYGNMSSACVFFILDEMRKKSDEDGAPTTGEGLDWGVLFGFGPGLTVETVVLHSLPTTMSIAT

SEQ ID NO: 8 - protein, 394 aa, Helicrysum umbraculigerum
MASSINISKIREAQRAQGPASILAVGTANPSNYEIQADFPDYYFRVTKSEHMADMKGTFQRMCDKSMIRKRHMLITEEFLKENPNLCEYMAPSLDTRQDVVVVEVPKLGKEAATKAIKEWGQPKSKITHLIFCTTTGVDMPGADYQLTKLLGLAPSVKRFMIYQQGCFAGGTVLRLAKDIAENNKGARVLAVCSEITAMSFRGPNDTHVDSLVGQALFGDGAAAVIVGSDPDLTTERPLFEIISAAQTILPNSEGAIDGHVREVGLTIHILKDVPVLISKNIEKALTQAFSPLGISDWNSIFWIVHPGGPAILDQVELKVGLKKEKMATSRHVLSEYGNMSSACVFFIMDEMRKRSAKGGARTTGEGLDWGVLFGFGPGLTVETVVLHSLPTTM